(19) United States
(12) Patent Application Publication
(10) Pub. No.: US 2026/0087628 A1
(43) Pub. Date: Mar. 26, 2026

(54) Radiology Imaging Data Management Pipeline for Artificial Intelligence Workflows

(71) Applicant: Bristol-Myers Squibb Company, Princeton, NJ (US)

(72) Inventors: Devansh Agarwal, Lawrenceville, NJ (US); Kshitij Aggarwal, Princeton, NJ (US)

(73) Assignee: Bristol-Myers Squibb Company, Princeton, NJ (US)

(21) Appl. No.: 19/339,565

(22) Filed: Sep. 25, 2025

Related U.S. Application Data
(60) Provisional application No. 63/699,561, filed on Sep. 26, 2024.

(51) Int. Cl. G06T 7/00; G06T 1/20; G06T 7/11; G06T 11/00; G06V 10/764; G06V 10/82; G06V 40/10; G16H 10/20; G16H 30/20; G16H 30/40

(52) U.S. Cl. CPC G06T 7/0014; G06T 1/20; G06T 7/11; G06T 12/30; G06V 10/764; G06V 40/10; G16H 10/20; G16H 30/20; G16H 30/40; G06T 2207/10081; G06T 2207/10088; G06T 2207/10104; G06T 2207/10132; G06T 2207/20084; G06T 2207/30061; G06T 2207/30096; G06T 2210/32; G06T 2210/41; G06V 10/82; G06V 2201/10

(57) ABSTRACT

A method includes receiving an image scan of a patient and extracting, from the image scan having a first image format, metadata. The method also includes standardizing the metadata extracted from the image scan having the first image format according to a format of a schema associated with a relational database, and storing the received image scan having the first image format in data storage and the standardized metadata in the relational database. The method also includes converting the image scan having the first image format into a corresponding image scan having a second image format different than the first image format, and storing the corresponding image scan having the second image format in the data storage.

### CROSS REFERENCE TO RELATED APPLICATIONS

This U.S. Patent application claims priority under 35 U.S.C. § 119 (e) to U.S. Provisional Application 63/699,561, filed on Sep. 26, 2024. The disclosure of this prior application is considered part of the disclosure of this application and is hereby incorporated by reference in its entirety.

### TECHNICAL FIELD

This disclosure relates to radiology imaging data management pipeline for artificial intelligence workflows.

### BACKGROUND

Clinical trial sites receive continuous streams of imaging data that includes image scans of tissue, tumor sites, and internal organs to name few. The imaging data is collected from labs or healthcare facilities and are typically in the Digital Imaging and Communications in Medicine (DICOM) image format. While the DICOM image format has revolutionized the radiology industry, encompassing many imaging modalities such as X-rays, computed tomography (CT), magnetic resonance imaging (MRI), ultrasound, nuclear medicine, and PET scans, DICOM image scans are extremely large in size. Due to the large size of each DICOM image scan compounded by the vast number of DICOM image scans when a clinical trial site is monitoring multiple clinical trials simultaneously, it becomes a daunting challenge for the clinical trial site to undertake systematic data logging, retrieval, and utilization. Conventional techniques rely on individual data scientists to manage the incoming DICOM image scans, however, these techniques are only practicable when managing DICOM image scans for a small number of clinical trials, and are not scalable for large volumes of data associated with multiple ongoing clinical trials. Additionally, the ongoing addition and modification of DICOM image scans by core labs requires a dynamic solution to ensure data integrity and accuracy.

### SUMMARY

One aspect of the disclosure provides a computer-implemented method that when executed on data processing hardware causes the data processing hardware to perform operations for managing, cataloging, and converting incoming image scans of patients participating in clinical trials. The operations include receiving an image scan of a patient. The received image scan has a first image format. The operations also include extracting, from the image scan having the first image format, metadata, standardizing the metadata extracted from the image scan having the first image format according to a format of a schema associated with a relational database, and storing the received image scan having the first image format in data storage and the standardized metadata in the relational database. The operations also include converting the image scan having the first image format into a corresponding image scan having a second image format different than the first image format, and storing the corresponding image scan having the second image format in the data storage.

Implementations of the disclosure may include one or more of the following optional features. In some implementations, the operations also include extracting, from the corresponding image scan having the second image format, metadata; standardizing the metadata extracted from the image scan having the second image format according to the format of the schema associated with the relational database; and storing the standardized metadata in the relational database. The schema associated with the relational database may include a clinical trial table that links to a subject table, a visit table that links to the subject table, a scan table that links to the visit table, a first type of image scan table that links to the scan table, and a second type of image scan table that links to the scan table.

In some examples, the image scan having the first image format is composed of a plurality of two-dimensional image slices, and the corresponding image scan having the second image format includes a three-dimensional image. In these examples, converting the image scan having the first image format into the corresponding image scan having the second image format further may further include compressing the corresponding image scan having the second image format prior to storing the corresponding image scan having the second image format in the data storage. Moreover, in these examples, the first image format may include a Digital Imaging and Communications in Medicine (DICOM) image format and the second image format may include a Neuroimaging Informatics Technology Initiative (NIFTI) image format.

The image scan having the first image format may include a greater size than the corresponding image scan having the second image format. As aforementioned, the first image format may include the DICOM image format. The operations may also include receiving a natural language prompt to access and/or retrieve at least one of the image scan having the first image format or the corresponding image scan having the second image format stored in the data storage, ingesting, by a sequence processing neural network, the schema associated with the relational database. Based on the schema ingested by the sequence processing neural network, the operations may also include structuring, by the sequence processing neural network model, the natural language prompt into a corresponding relational database prompt that includes the format of the schema, and prompting, using the relational database prompt, the relational database to access and/or retrieve the at least one of the image scan having the first image format or the corresponding image scan having the second image format stored in the data storage.

In some examples, the operations also include, after storing the corresponding image scan having the second image format in the data storage, processing, using an image segmentation model, the corresponding image scan having the second image format to generate an annotated version of the corresponding image scan having the second image format. Here, the annotated version of the corresponding image scan annotates particular body parts present within the corresponding image scan. In these examples, the operations may further include: extracting, from the annotated version of the corresponding image scan having the second image format, metadata indicating labels for the annotated particular body parts present within the corresponding image scan; standardizing the extracted metadata indicating the labels for the annotated particular body parts according to the format of the schema associated with the relational database; and storing the standardized extracted metadata indicating the labels for the annotated particular body parts in the relational database.

Another aspect of the disclosure provides a system that includes data processing hardware and memory hardware storing instructions that when executed on the data processing hardware causes the data processing hardware to perform operations for managing, cataloging, and converting incoming image scans of patients participating in clinical trials. The operations include receiving an image scan of a patient. The received image scan has a first image format. The operations also include extracting, from the image scan having the first image format, metadata, standardizing the metadata extracted from the image scan having the first image format according to a format of a schema associated with a relational database, and storing the received image scan having the first image format in data storage and the standardized metadata in the relational database. The operations also include converting the image scan having the first image format into a corresponding image scan having a second image format different than the first image format, and storing the corresponding image scan having the second image format in the data storage.

This aspect of the disclosure may include one or more of the following optional features. In some implementations, the operations also include extracting, from the corresponding image scan having the second image format, metadata; standardizing the metadata extracted from the image scan having the second image format according to the format of the schema associated with the relational database; and storing the standardized metadata in the relational database. The schema associated with the relational database may include a clinical trial table that links to a subject table, a visit table that links to the subject table, a scan table that links to the visit table, a first type of image scan table that links to the scan table, and a second type of image scan table that links to the scan table.

In some examples, the image scan having the first image format is composed of a plurality of two-dimensional image slices, and the corresponding image scan having the second image format includes a three-dimensional image. In these examples, converting the image scan having the first image format into the corresponding image scan having the second image format further may further include compressing the corresponding image scan having the second image format prior to storing the corresponding image scan having the second image format in the data storage. Moreover, in these examples, the first image format may include a Digital Imaging and Communications in Medicine (DICOM) image format and the second image format may include a Neuroimaging Informatics Technology Initiative (NIFTI) image format.

The image scan having the first image format may include a greater size than the corresponding image scan having the second image format. As aforementioned, the first image format may include the DICOM image format. The operations may also include receiving a natural language prompt to access and/or retrieve at least one of the image scan having the first image format or the corresponding image scan having the second image format stored in the data storage, ingesting, by a sequence processing neural network, the schema associated with the relational database. Based on the schema ingested by the sequence processing neural network, the operations may also include structuring, by the sequence processing neural network model, the natural language prompt into a corresponding relational database prompt that includes the format of the schema, and prompting, using the relational database prompt, the relational database to access and/or retrieve the at least one of the image scan having the first image format or the corresponding image scan having the second image format stored in the data storage.

In some examples, the operations also include, after storing the corresponding image scan having the second image format in the data storage, processing, using an image segmentation model, the corresponding image scan having the second image format to generate an annotated version of the corresponding image scan having the second image format. Here, the annotated version of the corresponding image scan annotates particular body parts present within the corresponding image scan. In these examples, the operations may further include: extracting, from the annotated version of the corresponding image scan having the second image format, metadata indicating labels for the annotated particular body parts present within the corresponding image scan; standardizing the extracted metadata indicating the labels for the annotated particular body parts according to the format of the schema associated with the relational database; and storing the standardized extracted metadata indicating the labels for the annotated particular body parts in the relational database.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for managing, cataloging, and converting incoming image scans of patients participating in clinical trials.

FIG. 2 is a schematic view of an example schema for metadata stored in a relational database of the system of FIG. 1

FIG. 3A is a schematic view of a client device issuing a natural language prompt to retrieve image scans for a particular patient from a particular clinical trial.

FIG. 3B is a schematic view of the client device of FIG. 3A providing the retrieved image scans to a tumor segmentation model 360 that is trained to detect and annotate a size of a tumor in the retrieved image scans over a duration of the particular clinical trial.

FIG. 4 is a flowchart of an example arrangement of operations for a method of managing, cataloging, and converting incoming image scans of patients participating in clinical trials.

FIG. 5 is a schematic view of an example computing device that may be used to implement the systems and methods described herein.

Like reference symbols in the various drawings indicate like elements.

### DETAILED DESCRIPTION

Clinical trial sponsors receive continuous streams of imaging data associated with clinical trials that includes image scans of tissue, tumor sites, and internal organs to name few. Imaging data is recorded as part of standard protocol in clinical trials for diagnosing, staging, and governing the treatment of diseases. The imaging data is collected from labs or healthcare facilities and are typically in the Digital Imaging and Communications in Medicine (DICOM) image format. While the DICOM image format has revolutionized the radiology industry, encompassing many imaging modalities such as X-rays, computed tomography (CT), magnetic resonance imaging (MRI), ultrasound, nuclear medicine, and PET scans, DICOM image scans are extremely large in size. Due to the large size of each DICOM image scan compounded by the vast number of DICOM image scans when a clinical trial site is monitoring multiple clinical trials simultaneously, it becomes a daunting challenge for the clinical trial site to undertake systematic data logging, retrieval, and utilization. Conventional techniques rely on individual data scientists to manage the incoming DICOM image scans; however, these techniques are only practicable when managing DICOM image scans for a small number of clinical trials and are not scalable for large volumes of data associated with multiple ongoing clinical trials. Moreover, not only are clinical trial sites receiving new imaging data, but the image data is continuously being updated by core labs. For instance, the core labs run quality checks and anonymize incoming data. Additionally, the ongoing addition and modification of DICOM image scans by core labs requires a dynamic solution to ensure data integrity and accuracy.

The DICOM image format stores the image data for each scan in two-dimensional slices, rendering the format unsuitable for training foundational machine learning models on the image data. By contrast, the Neuroimaging Informatics Technology Initiative (NIFTI) image format stores image data as a three-dimensional image and was designed to promote interoperability between software tools. As such, the NIFTI image format is useful for training foundational machine learning models.

Implementations herein are directed toward an automated system capable of effortlessly handling and updating millions of scans, enabling the ability to record each scan's details in data storage upon arrival at the clinical trial site for easy access. Specifically, the automated system automates cataloging the scans in a first image format (e.g., DICOM image format), converting the scans in the first image format to a second image format (e.g., NIFTI image format), extracting relevant data and metadata, and logging the extracted data and metadata to speed up the analysis process. The automated system provides a technological improvement by drastically reducing the time it takes individual data scientists to catalog the incoming scans, convert the scans from the first image format to the second image format, and extract relevant data from days to hours.

As will become apparent, the automated system disclosed herein drastically improves the ability for data scientists and researchers to handle medical imaging data from multiple clinical trials in a streamlined, systematic, and scalable manner. Moreover, the automated system fosters consistent data integrity, facilitates easy access and searching capabilities of the image scans and data extracted therefrom, and allows for a more efficient application of artificial intelligence and machine learning algorithms to be applied for building foundational models from the image scans as a direct result of the strategic conversion from the first (e.g., DICOM) image format to the second (e.g., NIFTI) image format. In addition to the drastic reduction in time and resources, the automated system also amplifies the potential for breakthroughs in disease (e.g., Cancer) treatment and research.

Generally, DICOM image scans contain large amounts of metadata that conveys the details about acquisition of the DICOM image scans as well as processing parameters. The automated system advantageously extracts and logs a subset of the metadata contained in the DICOM image scans for storage in a relational database. In addition, the automated system is capable of tracking any updates to an existing DICOM image scan in scenarios where the core labs modify a header of the DICOM image scan for accuracy and standardization. In some scenarios, the automated system will render an incoming DICOM image scan as being invalid if the underlying image data or corresponding metadata extracted therefrom is determined to be corrupted for any reason.

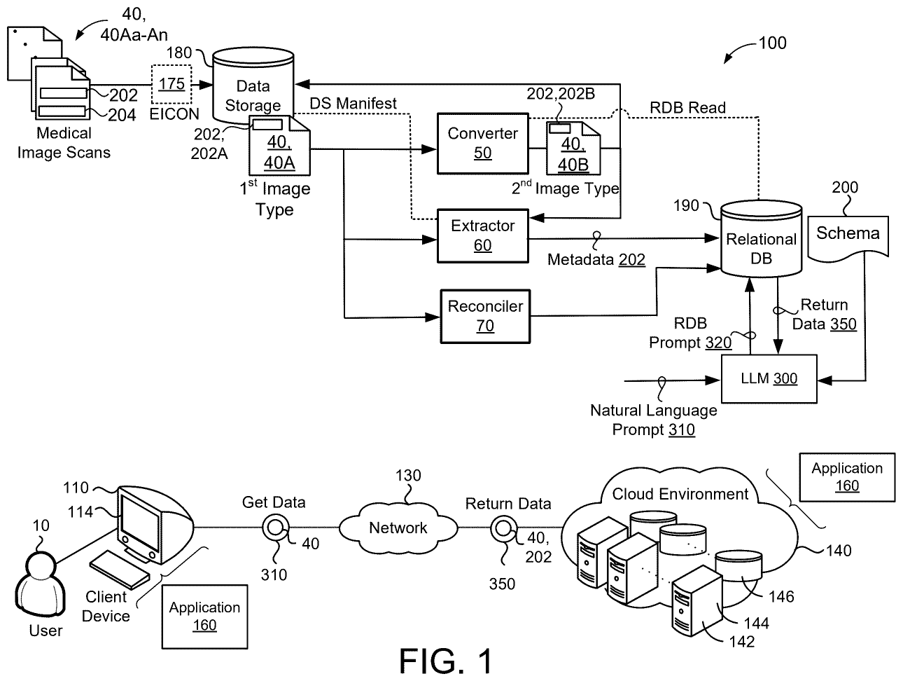

Referring to FIG. 1, in some implementations, a system 100 includes a client device 110 accessing medical imaging data 40 from multiple clinical trials via a medical imaging application 160 that logs and retrieves the medical imaging data in a streamlined, systematic, and scalable manner. The client device 110 is associated with a user 10 such as a data scientist or healthcare professional (HCP), who may communicate, via a network 130, with a remote system 140. The remote system 140 may be a distributed system (e.g., cloud environment) having scalable/elastic resources 142. The resources 142 include computing resources 144 (e.g., data processing hardware) and/or storage resources 146 (e.g., memory hardware). In some implementations, the remote system 140 executes the medical imaging application 160. Here, the client device 110 may access the application 160 running on the remote system 140 and input, via a graphical user interface (GUI) executing on the client device 110, a query/prompt 310 to access and/or retrieve medical imaging data 40 as well as metadata 202 associated with the medical imaging data 40 specified by the prompt 310. The prompt 310 may include a natural language prompt. As will be described in greater detail below, the medical imaging data 40 may be stored on data storage 180 overlain on the memory hardware 146 of the remote system 140, while the associated metadata 202 extracted from the medical imaging data 40 may be stored in a relational database 190 overlain on the memory hardware 146 of the remote system 140. The client device 110 may additionally or alternatively execute the application 160 to implement the ability to issue queries/prompts 310 for accessing/retrieving medical imaging data 40 and associated metadata 202 stored on the client device 110.

The system 100 receives medical imaging data 40 in the form of a first type of image scans 40, 40Aa-An from one or more core labs that acquired the scans from patients participating in clinical trials. While not shown, a portion of the medical imaging data 40 is in the form of a second type of image scans 40B. A portion of the image scans 40A may not be received from core labs and instead correspond to publicly available image scans. Optionally, the first type of image scans 40A may pass through an EICON board that tracks all incoming image scans and where the imaging scans came from (e.g., which core labs and which clinical trial sites collected the image scans). The system 100 may be managed and operated by a clinical trial sponsor. Each image scan 40A may include a scan of a body part of a respective patient participating in a clinical trial. The image scans 40A can include image scans of organs and tissues. Each image scan 40A may be initially obtained by an approved clinical trial site that provides the image scan 40A to the core lab, whereby the core lab performs quality checks on the image scan 40A and anonymizes the image scan 40A by removing any patient identifying information from the image scan 40A. For instance, the core lab may determine the image scan 40A is not properly labeled to indicate the body part the image scan 40A corresponds to and take corrective action so that the correct label is applied.

The incoming image scans 40A received from the core labs may be stored in the data storage 180 overlain on the storage resources 146 of the remote system 140 managed and operated by the clinical trial sponsor. The first type of image scan 40A may include a first image format. The first image format may include the image data for a scan of a body part as two-dimensional slices, rendering the format unsuitable for training or executing foundational machine learning models on the image data. In some examples, the first image format associated with the first type of image scans 40A includes the DICOM image format. The DICOM image format includes image scans in different modalities such as X-rays, computed tomography (CT), magnetic resonance imaging (MRI), ultrasound, nuclear medicine, and PET scans. DICOM image scans are extremely large in size. For simplicity, the present disclosure will refer to the first type of image scans 40A received from the core labs as DICOM image scans 40A. However, the first image format associated with the first type of image scans 40A may include image formats other than DICOM image scans with departing from the scope of the present disclosure. Due to the large size of each DICOM image scan compounded by the vast number of DICOM image scans when a clinical trial site is monitoring multiple clinical trials simultaneously, it becomes a daunting challenge for the clinical trial sponsor to undertake systematic data logging, retrieval, and utilization. Conventional techniques rely on individual data scientists to manage the incoming DICOM image scans; however, these techniques are only practicable when managing DICOM image scans for a small number of clinical trials and are not scalable for large volumes of data associated with multiple ongoing clinical trials. Moreover, not only are clinical trial sites receiving new imaging data, but the image data is continuously being updated by core labs.

The system 100 also includes a converter 50, an extractor 60, and a reconciler 70 that are each configured to process each DICOM image scan 40A received by the core labs and stored in the data storage 180 of the clinical trial site. Each DICOM image scan 40A includes associated metadata 202, 202A appended thereto. For each DICOM image scan 40A, the extractor 60 is configured to extract and store the metadata 202A in the relational database (RDB) 190. Here, the extractor 60 may standardize the extracted metadata 202A according to a schema 200 associated with the RDB 190 and then store the standardized metadata 202A in the RDB 190. The metadata 202A associated with DICOM image scan 40A may be standardized to include a format corresponding to the schema 200 that provides a trial identifier (id) indicating a name of the clinical trial associated with the DICOM image scan 40A, a patient id linked to the trial id that indicates an anonymized patient associated with the DICOM image scan 40A who is participating in the clinical trial, a visit id linked to the patient id that indicates a name and date of an underlying clinical trial visit (e.g., clinical trial site) at which the DICOM image scan 40A was obtained, and a scan id linked to the visit id that indicates a name of the DICOM image scan and other pertinent details such as a body region, modality, and scan plane of the DICOM image scan 40. Notably, each clinical trial has multiple patients, each patient has multiple clinical trial visits, and each clinical trial visit may obtain multiple DICOM image scans 40A for the underlying anonymized patient. The extractor 60 may seamlessly extract the metadata 202A from the DICOM image scans 40 associated with thousands of patients across a multitude of different clinical trials managed by the clinical trial sponsor. For the duration of a clinical trial, each patient may require a DICOM image scan 40A at predetermined determined time points (e.g., every four weeks).

Each DICOM image scan 40A may be composed of a multitude of two-dimensional image slices and include a total size of about 200 megabytes (mb). Accordingly, each two-dimensional image slice of the multitude of two-dimensional image slices of each DICOM image scan 40A may be saved and stored as a separate respective file. The converter 50 may perform a read on the relational database 190 to ascertain the underlying schema 200 applied by the relational database 190. For each DICOM image scan 40A, the converter 50 is configured to convert the DICOM image scan 40A into a corresponding second type of image scan 40B by stacking the multiple two-dimensional image slices of the DICOM image scan 40A into a three-dimensional image and then compress the three-dimensional image to provide the second type of image scan 40B having a reduced size. For instance, the second type of image scan 40B may include a second image format having a reduced size of about 20 to 30 mb compared to the size of about 200 mb for the DICOM image scan 40A. Notably, the second type of image scan 40B may be stored as a single file. In some examples, the second type of image scan 40B converted from the DICOM image scan 40A by the converter 50 includes the Neuroimaging Informatics Technology Initiative (NIFTI) image format. After converting the DICOM image scan 40A to the corresponding NIFTI image scan 40B, the converter 50 stores the NIFTI image scan 40B in the data storage 180 and the extractor 60 may extract metadata 202, 202B associated with the NIFTI image scan 40B. For instance, the extractor 60 may link the metadata 202B associated with the NIFTI image scan 40B to the metadata 202B of the corresponding DICOM image scan 40A the NIFTI image scan 40B was converted from. For simplicity, the present disclosure will refer to the second type of image scans 40B as NIFTI image scans 40B. However, the second image format associated with the second type of image scans 40B may include image formats other than NIFTI image scans with departing from the scope of the present disclosure.

In some examples, an image segmentation model trained to annotate body parts present within NIFTI image scans processes the NIFTI image scan 40B to generate an annotated version of the NIFTI image scan 40B. Here, the annotated version of the NIFTI image scan annotates particular body parts present within the corresponding image scan. The annotated version of the NIFTI image scan may include metadata indicating all the particular body parts present within the image scan. The relational database may update the metadata to indicate the names of the particular body parts present within the image scan.

Notably, the system 100 may be programmed such that the extractor 60 extracts, standardizes, and stores the metadata 202 of a particular DICOM image scan 40A independent from the converter 50 converting the particular DICOM image scan 40A into the corresponding NIFTI image scan 40B. As such, the particular DICOM image scan 40A can be logged and stored in the data storage 180 and its associated extracted and standardized metadata 202A may be stored in the RDB 190 even if the converter 50 fails to successfully convert the particular DICOM image scan 40A into the corresponding NIFTI image scan 40B. In some examples, each incoming DICOM image scan 40A is stored in the data storage 180 and the extractor 60 extracts, standardizes and stores the metadata 202A of the incoming DICOM image scan 40A before the converter 50 converts the DICOM image scan 40A into the NIFTI image scan 40B.

For each DICOM image scan 40A, the reconciler 70 performs reconciliation by pulling in data from ongoing clinical trials so that insights can be ascertained from the ongoing clinical trials. For instance, the multitude of patients participating in a respective clinical trial may be instructed to make clinical trials visits within predetermined time windows (e.g., every four weeks) so that the DICOM image scans 40A and other pertinent information can be collected. However, the exact time points at which each patient visits various across all the patients. As such, the reconciler 70 is tasked with keeping track of which data has been received and which data has not yet been received for each given patient participating in the respective clinical trial. For example, at some later time after a DICOM image scan 40A is received from the core labs and its metadata is extracted by the extractor 60, a scientist performing analysis on the DICOM image scan 40A may determine that the image scan 40Aincludes defects or has other issues that may render the image scan 40A invalid. Here, the application 160 may instruct the core labs to fix the invalid image scan 40A whereby the core lab will delete the initial DICOM image scan 40A and collect a new DICOM image scan 40A that fixes the identified defect. As such, the reconciler 70 may compare the DICOM image scan 40A stored in the data storage 180 that was deemed defective with the corresponding new DICOM image scan 40A received from the core labs that fixed the identified defect to reconcile what changes are present in the corresponding new DICOM image scan 40A. Here, the reconciler 70 will determine that the new DICOM image scan 40A is related to the defective DICOM image scan 40A because the two scans will be associated with the same patient id, same clinical trial visit, and the same date.

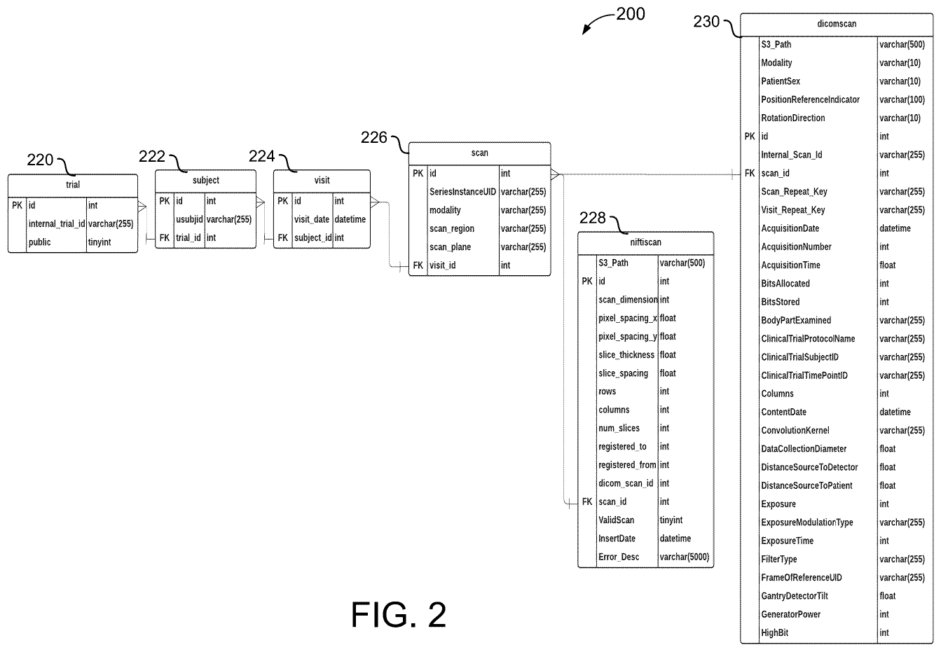

FIG. 2 shows an example of the schema 200 for the metadata 202 stored in the RDB 190. The schema 200 may include a plurality of tables that each link to one another. The tables and attributes for each table depicted by the schema 200 shown in FIG. 2 are only exemplary and the schema 200 may include additional attributes in each table as well as additional tables without departing from the scope of the present disclosure. In the example shown, the schema 200 includes a trial table 220, a subject table 222, a visit table 224, a scan table 226, a first type of scan table 228, and a second type of image scan table 230. Here, the trial table 220 includes a primary key (PK) for a trial id indicating a name of the trial, as well as other parts including an internal trial id and public. The subject table 222 includes a PK for an anonymized patient id and the trial id linked to the trial table 220 by a foreign key (FK). The visit table 224 includes a PK for the clinical trial visit id, as well as other parts including a visit date and the anonymized patient id linked to the subject table 222 by an FK.

The scan table 226 includes a scan id that indicates the name of the related image scans 40 obtained from the patient as a PK, a series instance of the scan, a modality of the scan, a scan region, a scan plane, and the visit id that links to the visit table 224 by an FK. The scan id associated with the PK of the scan table 226 links to each of a DICOM scan table 230 and a NIFTI image scan table 228 associated with respective ones of an original DICOM image scan 40A received from the core labs and the corresponding NIFTI image scan 40B converted from the original DICOM image scan 40A by the converter 50.

The metadata 202 of the DICOM image scan table 230 includes a data storage path indicating a location at which the respective DICOM image scan 40A is stored in the data storage 180, the modality of the image scan 40A, a patient sex, position reference indicator, rotation direction, a DICOM scan id as a PK, an internal scan id, a scan repeat key, a visit repeat key, an acquisition date, an acquisition number, an acquisition time, bits allocated, bits stored, body part examined, clinical trial protocol/name, clinical trial subject id, clinical trial time point id, columns, content date, convolution kernel, data collection diameter, distance source to detector, distance source to detector, exposure, exposure modulation type, exposure time, filter type, frame of reference UID, gantry detector tilt, generator power, and high bit.

The metadata 202 of the NIFTI image scan table 228 includes a data storage path indicating a location at which the respective NIFTI image scan 40B is stored in the data storage 180, a NIFTI scan id as a PK, scan dimension, pixel spacing along x-dimension, pixel spacing along y-dimension, slice thickness, slice spacing, rows, columns, number of slices, registered to, registered from, the DICOM scan id, the scan id that links to the scan table by a FK, valid scan, and insert date. The number of slices, slice thickness, and slice spacing may correspond to the slices of the DICOM image scan 40A the respective NIFTI image scan 40A was converted from.

Referring back to FIG. 1, in some implementations, the system 100 includes a a sequence processing neural network 300 executing on the data processing hardware 144 of the remote system 140 and/or locally on the client device 110. The user 10, such as a data scientist or HCP, may access the sequence processing neural network 300 via the application 160 executing on the client device 110 to issue a query/prompt 310 to access and/or retrieve medical imaging data 40 as well as metadata 202 associated with the medical imaging data 40 specified by the prompt 310. The user 10 may access the application 160 and sequence processing neural network 300 via the GUI executing on the client device 110 and issue the prompt 310 via any combination of spoken input or typed input. In scenarios when the user provides the prompt via spoken input, the client device 110 may employ a microphone to record audio data characterizing the spoken input of the prompt 310 and the application 160 may leverage, i.e., via an API, a speech recognition system configured to convert the audio data characterizing the spoken input into a textual representation of the prompt 310. In other scenarios, the user 10 may provide the typed input of the prompt via a keyboard (physical or virtual) in communication with the GUI executing on the client device 110. The GUI may be displayed on a screen 114 of the client device 110.

In some examples, the prompt 310 includes a natural language prompt that specifies specific medical imaging data 40 stored in the data storage 180 and/or metadata 202 stored in the RDB 190 that the user 10 wants to access and/or retrieve. For example, the natural language prompt 310 issued by the user 10 may include the utterance “Show me all CT scans after Sep. 6, 2024”. Notably, since the RDB 190 stores the metadata 202 that is standardized to include the format of the schema 200 (e.g., see FIG. 2) used by the RDB, the sequence processing neural network 300 is configured to operate as an intermediary between the client device 110 and the RDB 190 by structuring the natural language prompt 310 into a corresponding RDB prompt 320 that includes the format of the schema 200. In these examples, the sequence processing neural network 300 ingests the schema 200 of the RDB 190 and processes the natural language prompt 310 to structure the RDB prompt 320 for prompting the RDB 190 to access and/or retrieve the medical imaging data 40 and/or associated metadata 202 specified by the natural language prompt 320. Here, and continuing with the example, the sequence processing neural network 300 may process the natural language prompt 310 and apply the schema 200 to determine to use the modality parameter of the DICOM image scan table 230 for “CT scans” and the acquisition date parameter of the DICOM image scan table 230 for “dates after Sep. 6, 2024”. Based on the RDB prompt 320 input to the RDB 190, the RDG 190 provides return data 350 back to the client device 110 that includes the medical imaging data 40 and/or associated metadata 202 specified by the natural language prompt 310. For instance, the RDB 190 may process the RDB prompt 320 to retrieve, from the corresponding data storage locations of the data storage 180, all DICOM image scans 40A in the CT modality that were obtained for patients after Sep. 6, 2024 and the sequence processing neural network 300 may return the retrieved DICOM image scans 40A to the client device 110 as corresponding return data 340. Thereafter, the GUI executing on the client device 110 may graphically display the retrieved DICOM image scans 40A on the screen 114 of the client device 110.

The sequence processing neural network 300 may include a large language model (LLM) 300. For simplicity, the present disclosure will refer to the sequence processing neural network 300 as an LLM 300. However, the sequence processing neural network 300 may include other types of sequence processing neural networks other than LLMs 300 that are capable ingesting a schema 200 and structuring a natural language prompt 310 into a corresponding RDB prompt 320 that includes a format of the ingested schema 200.

In some implementations, the user 10 issues a natural language prompt 310 to the LLM 300 to identify all DICOM image scans 40A that are not paired with corresponding NIFTI image scans 40B and to convert the identified DICOM image scans 40A into the corresponding NIFTI image scans 40B. In these implementations, the LLM 300 may structure the natural language prompt 310 into the suitable RDB prompt 320 that applies the schema 200 to instruct the RDB 190 to identify each DICOM image scan 40A stored in the data storage 180 that is not paired with a corresponding NIFTI image scan 40B cause the converter 50 to convert each identified DICOM image scan 40A into the corresponding NIFTI image scan 40B. Here, each NIFTI image scan 40B converted from the identified DICOM image scans 40A may be stored in the data storage 180 and the extractor 60 may extract, standardize, and store the associated metadata 202B in the RDB 190.

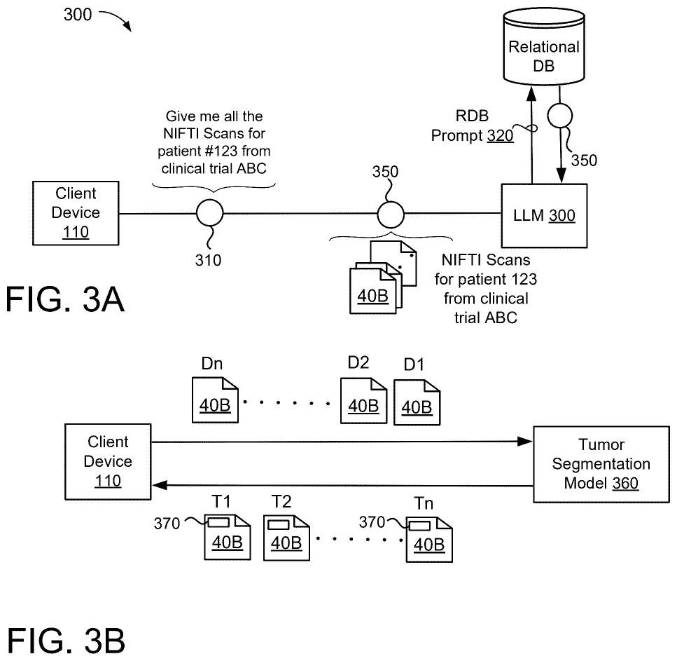

FIGS. 3A and 3B depict an example use case of the client device 110 issuing a natural language prompt 310 (FIG. 3A) from the user 10 to retrieve all NIFTI image scans 40B for a particular patient from a particular clinical trial and providing (FIG. 3B) the retrieved NIFTI image scans 40B to a tumor segmentation model 360 that is trained to detect and annotate the size of a tumor in the retrieved NIFTI image scans 40B over the duration of the particular clinical trial. As shown in FIG. 3A, the client device 110 may issue the natural language prompt 310 that includes the utterance “Give me the list of all the NIFTI Scans for patient #123 from clinical trial ABC”. Here, the user 10 (e.g., data scientist or HCP) may be interested in how a particular patient diagnosed with lung cancer responded to the therapy/treatment associated with the clinical trial ABC123. As mentioned previously, all incoming DICOM image scans 40A initially obtained by an approved clinical trial site during prescribed clinical visits are anonymized by the core labs by removing patient identifying information. Accordingly, the core labs may assign a unique anonymized patient identifier to the DICOM image scans 40A, whereby the extractor 60 extracts and standardizes the anonymized patient identifier as metadata 202 for storage in the DICOM image scan table 230 of the RDB 190.

The LLM 300 may ingest the schema 200 of the RDB 190 and process the natural language prompt 310 to structure an RDB prompt 320 for prompting the RDB 190 to retrieve all NIFTI image scans 40B obtained for patient #123 from the clinical trial ABC. As such, the LLM 300 may process the natural language prompt 310 and apply the schema 200 to determine the trial id parameter of the trial table 220 for “clinical trial ABC” and the patient id parameter of the subject table 222 for “patient #123” for use in structuring the RDB prompt 320 to receive all the NIFTI image scans 40B for the patient #123 that were converted from the original DICOM image scans 40A obtained from each clinical trial visit of the patient during the duration of clinical trial ABC. Based on the RDB prompt 320 input to the RDB 190, FIG. 3A shows the RDB 190 providing return data 350 that includes a plurality of NIFTI image scans 40B for the patient #123 each associated with a respective date of a respective clinical trial visit at which the corresponding DICOM image scan 40A was obtained. For instance, the plurality of NIFTI image scans 40B are each associated with a respective acquisition date ranging from date D1 to Dn. Patients participating in clinical trial ABC may be instructed to make clinical trial visits during predetermined time windows over the duration of the clinical trial to obtain DICOM image scans 40A. That is, the NIFTI image scan 40A obtained for patient #123 at date D1 may be associated with a scan of the patient's lung when the patient #123 commenced the treatment/therapy associated with clinical trial ABC and date Dn may be associated with a most recent scan of the patient's lung during the clinical trial ABC or a last scan of the patient's lung after the last treatment/therapy associated with the clinical trial ABC.

As shown in FIG. 3B, the client device may provide, as input, the NIFTI image scans 40B for the patient #123 at each of the acquisition dates D1-DN to the tumor segmentation model 360. Here, the tumor segmentation model 360 processes each NIFTI image scan 40B to detect and annotate the presence of the lung tumor in the corresponding NIFTI image scan 40B. The tumor segmentation model 360 may return each NIFTI image scan 40B back to the client device 110 with a respective annotation 370 of the lung tumor. In some examples, the annotation 370 of the lung tumor is depicted as a graphical overlay highlighting an area of the lung tumor within each NIFTI image scan 40B and/or a graphical overlay that outlines a perimeter of the lung tumor within each NIFTI image scan 40B. Additionally or alternatively, the tumor segmentation model 360 may be further trained to calculate other parameters after detecting and annotating the tumor in each NIFTI image scan 40B such as calculating a size of the lung tumor. Here, the client device 110 may graphically display, via the GUI, the annotated NIFTI image scans 40B obtained for the anonymized patient #123 at each of the acquisition dates D1-Dn so that the user 10 can visually see how the size of the tumor increased/decreased and the rate at which the tumor increased/decreased in response to the underlying treatment/therapy over the duration of the clinical trial. Without departing from the scope of the present disclosure, the user 10 may issue additional natural language prompts 310 to retrieve NIFTI image scans obtained for other anonymized patients diagnosed with lung cancer that participated in another clinical trial associated with a different treatment/therapy. As such, the tumor segmentation model 360 may similarly annotate those NIFTI image scans so that the user 10 can compare tumor growth in response to the different treatment/therapies.

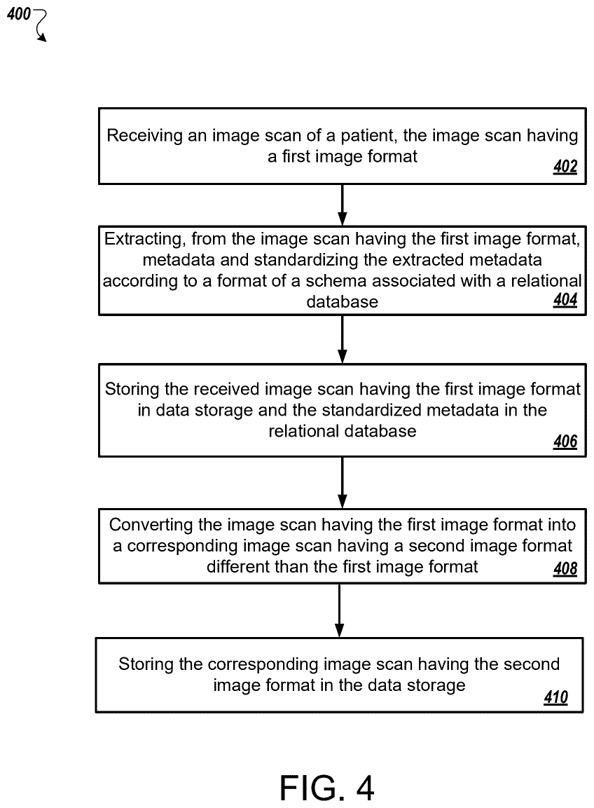

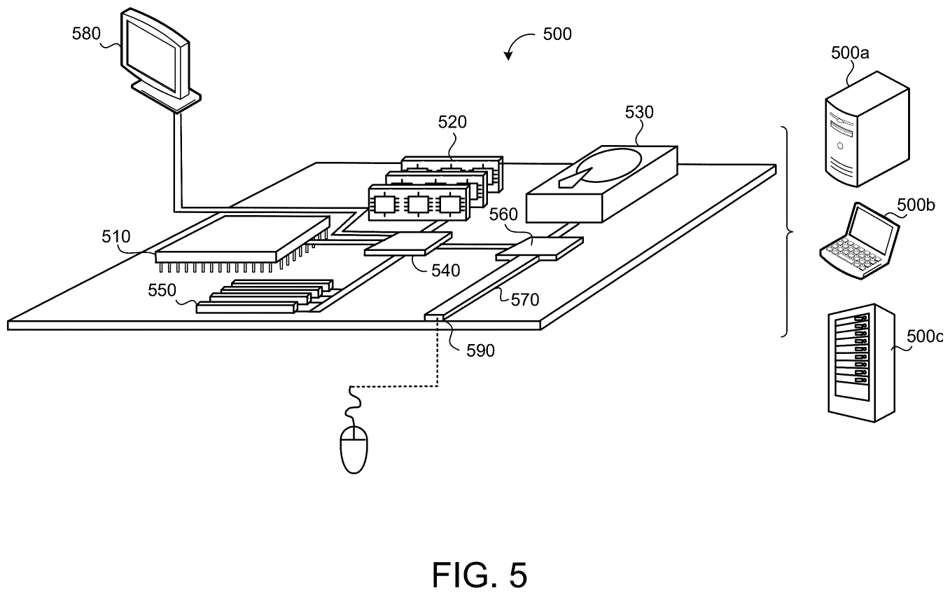

FIG. 4 provides a flowchart of an example arrangement of operations for a method 400 of managing, cataloging, and converting incoming image scans of patients participating in clinical trials. The method 400 may execute on data processing hardware 510 (FIG. 5) based on instructions stored on memory hardware 520 (FIG. 5) that cause the data processing hardware 510 to perform the operations. The data processing hardware 510 and the memory hardware 520 may include the data processing hardware 144 and the memory hardware 146 of the remote system 140. Additionally or alternatively, the data processing hardware 510 and the memory hardware 520 may reside on the client device 110.

At operation 402, the method 400 includes receiving an image scan 40A of a patient, the received image scan 40 having a first image format 40A. At operation 404, the method 400 includes extracting, from the image scan 40 having the first image format 40A, metadata 202 and standardizing the metadata 202 extracted from the image scan having the first image format 400 according to a format of a schema 200 associated with a relational database 190

At operation 406, the method 400 includes storing the received image scan having the first image format 40A in data storage 180 and the standardized metadata 202 in the relational database 190. At operation 408, the method 400 includes converting the image scan having the first image format 40A into a corresponding image scan having a second image format 40B different than the first image format. At operation 410, the method 400 includes storing the corresponding image scan having the second image format 40B in the data storage 180.

FIG. 5 is schematic view of an example computing device 500 that may be used to implement the systems and methods described in this document. The computing device 500 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 500 includes a processor 510, memory 520, a storage device 530, a high-speed interface/controller 540 connecting to the memory 520 and high-speed expansion ports 550, and a low speed interface/controller 560 connecting to a low speed bus 570 and a storage device 530. Each of the components 510, 520, 530, 540, 550, and 560, are interconnected using various busses, and may be mounted on a common motherboard or in other manners as appropriate. The processor 510 can process instructions for execution within the computing device 500, including instructions stored in the memory 520 or on the storage device 530 to display graphical information for a graphical user interface (GUI) on an external input/output device, such as display 580 coupled to high speed interface 540. In other implementations, multiple processors and/or multiple buses may be used, as appropriate, along with multiple memories and types of memory. Also, multiple computing devices 500 may be connected, with each device providing portions of the necessary operations (e.g., as a server bank, a group of blade servers, or a multi-processor system).

The memory 520 stores information non-transitorily within the computing device 500. The memory 520 may be a computer-readable medium, a volatile memory unit(s), or non-volatile memory unit(s). The non-transitory memory 520 may be physical devices used to store programs (e.g., sequences of instructions) or data (e.g., program state information) on a temporary or permanent basis for use by the computing device 500. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 530 is capable of providing mass storage for the computing device 500. In some implementations, the storage device 530 is a computer-readable medium. In various different implementations, the storage device 530 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer-or machine-readable medium, such as the memory 520, the storage device 530, or memory on processor 510.

The high speed controller 540 manages bandwidth-intensive operations for the computing device 500, while the low speed controller 560 manages lower bandwidth-intensive operations. Such allocation of duties is exemplary only. In some implementations, the high-speed controller 540 is coupled to the memory 520, the display 580 (e.g., through a graphics processor or accelerator), and to the high-speed expansion ports 550, which may accept various expansion cards (not shown). In some implementations, the low-speed controller 560 is coupled to the storage device 530 and a low-speed expansion port 590. The low-speed expansion port 590, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 500a or multiple times in a group of such servers 500a, as a laptop computer 500b, or as part of a rack server system 500c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving an image scan of a patient, the received image scan having a first image format;
extracting, from the image scan having the first image format, metadata;
standardizing the metadata extracted from the image scan having the first image format according to a format of a schema associated with a relational database;
storing the received image scan having the first image format in data storage and the standardized metadata in the relational database;
converting the image scan having the first image format into a corresponding image scan having a second image format different than the first image format; and
storing the corresponding image scan having the second image format in the data storage.

2. The method of claim 1, wherein the operations further comprise:
extracting, from the corresponding image scan having the second image format, metadata;
standardizing the metadata extracted from the image scan having the second image format according to the format of the schema associated with the relational database; and
storing the standardized metadata in the relational database.

3. The method of claim 1, wherein the schema associated with the relational database includes a clinical trial table that links to a subject table, a visit table that links to the subject table, a scan table that links to the visit table, a first type of image scan table that links to the scan table, and a second type of image scan table that links to the scan table.

4. The method of claim 1, wherein:
the image scan having the first image format is composed of a plurality of two-dimensional image slices; and
the corresponding image scan having the second image format comprises a three-dimensional image.

5. The method of claim 4, wherein converting the image scan having the first image format into the corresponding image scan having the second image format further comprises compressing the corresponding image scan having the second image format prior to storing the corresponding image scan having the second image format in the data storage.

6. The method of claim 4, wherein:
the first image format comprises a Digital Imaging and Communications in Medicine (DICOM) image format; and
the second image format comprises a Neuroimaging Informatics Technology Initiative (NIFTI) image format

7. The method of claim 1, wherein the image scan having the first image format comprises a greater size than the corresponding image scan having the second image format.

8. The method of claim 1, wherein the first image format comprises a Digital Imaging and Communications in Medicine (DICOM) image format.

9. The method of claim 1, wherein the operations further comprise, after storing the corresponding image scan having the second image format in the data storage, processing, using an image segmentation model, the corresponding image scan having the second image format to generate an annotated version of the corresponding image scan having the second image format, the annotated version of the corresponding image scan annotating particular body parts present within the corresponding image scan.

10. The method of claim 9, wherein the operations further comprise:
extracting, from the annotated version of the corresponding image scan having the second image format, metadata indicating labels for the annotated particular body parts present within the corresponding image scan;
standardizing the extracted metadata indicating the labels for the annotated particular body parts according to the format of the schema associated with the relational database; and
storing the standardized extracted metadata indicating the labels for the annotated particular body parts in the relational database.

11. The method of claim 1, wherein the operations further comprise:
receiving a natural language prompt to access and/or retrieve at least one of the image scan having the first image format or the corresponding image scan having the second image format stored in the data storage;
ingesting, by a sequence processing neural network, the schema associated with the relational database;
based on the schema ingested by the sequence processing neural network, structuring, by the sequence processing neural network, the natural language prompt into a corresponding relational database prompt that includes the format of the schema; and
prompting, using the relational database prompt, the relational database to access and/or retrieve the at least one of the image scan having the first image format or the corresponding image scan having the second image format stored in the data storage.

12. A system comprising:
data processing hardware; and
memory hardware in communication with the data processing hardware, the memory hardware storing instructions that when executed on the data processing hardware cause the data processing hardware to perform operations comprising:
receiving an image scan of a patient, the received image scan having a first image format;
extracting, from the image scan having the first image format, metadata;
standardizing the metadata extracted from the image scan having the first image format according to a format of a schema associated with a relational database;
storing the received image scan having the first image format in data storage and the standardized metadata in the relational database;
converting the image scan having the first image format into a corresponding image scan having a second image format different than the first image format; and
storing the corresponding image scan having the second image format in the data storage.

13. The system of claim 12, wherein the operations further comprise:
extracting, from the corresponding image scan having the second image format, metadata;
standardizing the metadata extracted from the image scan having the second image format according to the format of the schema associated with the relational database; and
storing the standardized metadata in the relational database.

14. The system of claim 12, wherein the schema associated with the relational database includes a clinical trial table that links to a subject table, a visit table that links to the subject table, a scan table that links to the visit table, a first type of image scan table that links to the scan table, and a second type of image scan table that links to the scan table.

15. The system of claim 12, wherein:
the image scan having the first image format is composed of a plurality of two-dimensional image slices; and
the corresponding image scan having the second image format comprises a three-dimensional image.

16. The system of claim 15, wherein converting the image scan having the first image format into the corresponding image scan having the second image format further comprises compressing the corresponding image scan having the second image format prior to storing the corresponding image scan having the second image format in the data storage.

17. The system of claim 15, wherein:
the first image format comprises a Digital Imaging and Communications in Medicine (DICOM) image format; and
the second image format comprises a Neuroimaging Informatics Technology Initiative (NIFTI) image format

18. The system of claim 12, wherein the image scan having the first image format comprises a greater size than the corresponding image scan having the second image format.

19. The system of claim 12, wherein the first image format comprises a Digital Imaging and Communications in Medicine (DICOM) image format.

20. The system of claim 12, wherein the operations further comprise, after storing the corresponding image scan having the second image format in the data storage, processing, using an image segmentation model, the corresponding image scan having the second image format to generate an annotated version of the corresponding image scan having the second image format, the annotated version of the corresponding image scan annotating particular body parts present within the corresponding image scan.

21. The system of claim 20, wherein the operations further comprise:
extracting, from the annotated version of the corresponding image scan having the second image format, metadata indicating labels for the annotated particular body parts present within the corresponding image scan;
standardizing the extracted metadata indicating the labels for the annotated particular body parts according to the format of the schema associated with the relational database; and
storing the standardized extracted metadata indicating the labels for the annotated particular body parts in the relational database.

22. The system of claim 12, wherein the operations further comprise:
receiving a natural language prompt to access and/or retrieve at least one of the image scan having the first image format or the corresponding image scan having the second image format stored in the data storage;
ingesting, by a sequence processing neural network, the schema associated with the relational database;
based on the schema ingested by the sequence processing neural network, structuring, by the sequence processing neural network, the natural language prompt into a corresponding relational database prompt that includes the format of the schema; and
prompting, using the relational database prompt, the relational database to access and/or retrieve the at least one of the image scan having the first image format or the corresponding image scan having the second image format stored in the data storage.
